(19) United States
(12) Patent Application Publication
(10) Pub. No.: US 2003/0013639 A1
(43) Pub. Date: Jan. 16, 2003

(54) Sleep inducing antacid composition

(76) Inventors: Lisa Yurchak, Green Brook, NJ (US); Robert Dobberstein, Lincoln, NE (US); Linda S. Heist, Lincoln, NE (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/898,155

(22) Filed: Jul. 3, 2001

(51) Int. Cl. A61K 38/17; A61K 31/7016; A61K 33/24; A61K 33/08; A61K 31/28

(52) U.S. Cl. 514/2; 424/653; 424/690; 514/53; 514/492

(57) ABSTRACT

The present invention relates to an orally administered sleep inducing antacid composition comprising from about 100 mg to about 2000 mg of at least one antacid, and about 300 mg to about 1000 mg of at least one sleep inducing compound wherein the amount of sleep inducing compound is based on a concentrated extract containing not less than 0.5% of the essential oil of the respective sleep inducing compound. The advantage of the sleep inducing antacid compositions are that they promote drowsiness and provide relief from excess gastrointestinal acidity.

### FIELD OF THE INVENTION

The present invention relates to an orally administered sleep inducing antacid composition comprising an antacid and sleep inducing compound. The advantage of the sleep inducing antacid compositions are that they promote drowsiness and provide relief from excess gastrointestinal acidity.

### BACKGROUND OF THE INVENTION

Antacid compositions are well known in the art and are used to treat a wide range of disorders such as upset stomach, heartburn, acid/or nervous indigestion, excess gastrointestinal acidity, reflux esophagitis, and ulcers. A common antacid, especially in solid form, is calcium carbonate. For example, calcium carbonate is the active ingredient in MAALOX quick dissolve antacid tablets, which are available from Novartis.

Recently, it has become increasingly common for people inflicted with such disorders which are mitigated by treatment with antacids, to experience episodes of such disorders at or near bedtime. While there are numerous commercially available antacid compositions which provide relief from excess gastrointestinal acidity, none of the currently available antacid compositions assist the user in sleeping.

### SUMMARY OF THE INVENTION

The present invention provides an orally administered sleep inducing antacid composition comprising from about 100 mg to about 2000 mg of at least one antacid, and about 300 mg to about 1000 mg of at least one sleep inducing compound wherein the amount of sleep inducing compound is based on a concentrated extract containing not less than 0.5% of the essential oil of the respective sleep inducing compound.

The advantage of the sleep inducing antacid compositions are that they promote drowsiness and provide relief from excess gastrointestinal acidity.

### DESCRIPTION OF THE INVENTION

The sleep inducing antacid composition of the invention are orally administered to humans or animals in solid or liquid form. The sleep inducing antacid composition are not harmful and generally recognized as safe (GRAS) and may be administered to children including babies. Suitable liquid forms include, but are not limited to, solutions, emulsions, and suspensions. Suitable solid and semi-solid forms include, but are not limited to, capsules, caplets, powders, and tablets, and include soft and hard, chewable and non-chewable forms.

The sleep inducing antacid composition contains an antacid and a sleep inducing compound. Examples of antacids suitable for use herein are any antacids acceptable to the Food and Drug Administration, such as aluminum carbonate, aluminum hydroxide (or as aluminum hydroxide-hexitol stabilized polymer, aluminum hydroxide-magnesium hydroxide codried gel, aluminum hydroxide-magnesium trisilicate codried gel, aluminum hydroxide-sucrose powder hydrated), aluminum phosphate, aluminum hydroxy carbonate, dihydroxy aluminum sodium carbonate, aluminum magnesium glycinate, dihydroxy aluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, magnesium trisilicate, sodium bicarbonate, potassium bicarbonate, glycine, dried milk solids, sodium carbonate, potassium carbonate, and sodium potassium tartrate. A combination of antacids may also be used. Preferably, the antacid is selected from aluminum hydroxide, calcium carbonate, magnesium carbonate, and magnesium hydroxide.

The antacid is present in the sleep inducing antacid composition in an amount to neutralize at least 5 milliequivalents of gastrointestinal acid. Preferably, the antacid is present in an amount of from about 100 mg to about 2000 mg, more preferably from about 200 mg to about 1200 mg. Most preferably, the antacid is present in an amount of from about 400 to about 800 mg.

The sleep inducing compound is selected from herbal compounds and nonherbal compounds which exhibit anxiety reducing activity. The herbal compound may be in the form of ground plant or parts of plant, liquid extract of plant part, a semi-solid of plant part, and/or a powder extract of plant part. Examples of sleep inducing compounds include melatonin, L-tryptophan, Amanita Muscaria (aga), Apium Graveolens (celery), Aquilegia Vulgaris (columbine), Artemisia Vulgaris (mugwort), Atropa Belladonna (belladonna), Avena Sativa (oats), Ballota Nigra (horehound), Betonica Officinalis (wood betony), Culluna Vulgaris (heather), Cannabis Sativa (marijuana), Capsella Bursa Pastoris (shepherd's purse), Chamaemelum Nobile (Roman chamomile), Cinnamomum Camphora (camphor tree), Citrus Aurantium (bitter orange), Convallaria Majalis (lily-of-the-valley), Corydalis Caba (corydalis), Cyclamen Europaeum (cyclamen), Cypripedium Calceolus (nerve root), Cytisus Scoparius (broom), Drimia Maritima (squill), Eschscholtzia Californica (California poppy), Fragaria Vesca (strawberry leaf), Galium Odorata (woodruff), Humulus Lupulus (hops), Hypericum Perforatum (St. John's wort), Ilex Paraguariensis (mate), Lavandula Angustifolia (lavender), Leonurus Cardiaca (motherwort), Lycopus Virginicus (bugleweed), Matricaria Chamomile L. (German chamomile), Matricaria recutita L. (Hungarian chamomile), Melissa Officinalis (lemon balm), Nepeta Cataria (catnip), Paris Quadrifolia (herb Paris), Passiflora Incarnata (passion flower), Piper Methysticum (kava-kava), Piscidia Piscipula (Jamaica dogwood), Primula Elatior (primrose), Prunus Serotina (black cherry), Rauwolfia Serpentina (rauwolfia), Scutellaria Lateriflora (scullcap), Selenicereus Grandiflorus (night-blooming cereus), Strychnos Nux Vomica (nux vomica), Syzygium Cumini (jambolan), Valeriana Officinalis (valerian), Verbena Officinalis (vervain), Veronica Officinalis (speedwell), and Viscum Album (mistletoe). A combination of sleep inducing compounds may also be used.

Preferably, the sleep inducing compound is selected from Apium Graveolens (celery), Avena Sativa (oats) Chamaemelum Nobile (Roman chamomile), Convallaria Majalis (lily-of-the-valley), Cypripedium Calceolus (nerve root), Hypericum Perforatum (St. John's wort) Lavandula Angustifolia (lavender), Mattricaria Chamomile L. (German chamomile), Nepeta Cataria (catnip), Piper Methysticum (kava-kava), and Valeriana Officinalis (valerian). Most preferably, the sleep inducing compound is valerian or kava-kava.

As used herein, valerian includes any of the fresh, dried, ground, or powdered parts of Valeriana Officinalis Linne, such as the rhizome, roots, and stolons, any extract or volatile oil obtained from Valeriana Officinalis, and any compounds collectively referred to as valerenic acids. Examples of compounds collectively referred to as valerenic acids are valerenic acid, hydroxyvalerenic acid, acetoxyvalerenic acid, valerenal, valeranone, kessyl glycol, and valepotriates, such as didrovaltrate, valtrate, and isovaltrate. As used herein, kava-kava includes any of the fresh or dried, ground or powdered parts of Piper Methysticum G. Forster, such as the rhizomes and roots, any extract obtained from Piper Methysticum, and any compounds referred collectively as kavalactones or kava pyrones, such as methysticin, dihydromethysticin, kawain, dihydrokawain, yangonin, marindinin, and desmethoxyyangonin.

The amount of sleep inducing compound in the sleep inducing antacid composition is preferably from about 300 mg to about 1000 mg based on concentrated or solid extract containing not less than 0.5% of the essential oil of the respective sleep inducing compound. More preferably, the sleep inducing compound is present in an amount of from about 400 mg to about 860 mg, and most preferably, in an amount of from about 430 mg to about 600 mg.

A food and/or pharmaceutical processing aid is optionally included in the sleep inducing antacid composition to assist in binding all components together, and thereby ensure final product texture, consistency, and, if applicable, dispersability in aqueous media. Examples of processing aids include phosphatides, phospholipids, gelatins, gums such as gum arabic, carrageenan, guar gum, locust bean gum, pectins, and cellulose derivatives. Other food processing aids include those with desirable wetting, lubricating, emulsifying, gelling, swelling, or penetrating properties. Of these, lecithin, either alone or in combination with one or more gums or gelatins may be desirable.

Additional ingredients which may be added to the sleep inducing antacid composition include natural and/or artificial ingredients such as vitamins, botanicals, supplements, minerals, trace elements, amino acids (e.g., L. tryptophan), antiflatulents such as simethicone, herbs, fiber, flavorants, enzymes, fillers, buffers, colorants, dyes, sweeteners, pharmaceutical active compounds, antioxidants, medicaments, preservatives, electrolytes, glidants, disintegrates, lubricants, and carrier materials such as polysaccharides. A combination of additional ingredients may also be used. Such ingredients are known to those skilled in the art and preferably are used in the sleep inducing antacid composition in an amount that corresponds to an amount generally recognized as both safe and effective by the United States Food & Drug Administration. For those additional ingredients for which no RDA and/or DV (daily value) has been officially promulgated, then an amount generally accepted in the art as both safe and efficacious may be utilized.

The vitamins are preferably those that are directed to the overall reduction of stress of the user. Accordingly, the vitamin B complex may be selected. This group preferably includes thiamine (B₁) (available as thiamine hydrochloride and thiamine mononitrate), riboflavin (B₂), different chemical forms of what is now considered to be B₃, different chemical forms of what is now considered to be B₅, pyridoxine HCl (B₆) and cyanocobalamin (B₁₂).

Other vitamins may be selected such as vitamin C, E, or B₁₂. It is noted that the antineuritic vitamin thiamine has particular application in reducing emotional hypersensitivity, muscular weakness and fatigue. Riboflavin is important in tissue respiration. Pyridoxine is essential for the dehydration and desulfydration of amino acids and for the normal metabolism of tryptophan. It also appears to be related to fat metabolism. Beneficial properties have also been attributed to the use of folic acid.

Examples of botanicals are Zingiber officinale Roscoe (ginger), tiana lutea (gentianaceae or gentian), Centaurium erythrae Rafn (centaury), Swertia chirata (bitterstick), Menyanthes trifoliata (bogbean), Artermisia absinthium (Worm wood), Rubis fruiticousus (blackberry leaves or fruit), Rubis family (Blackberry root), Vaccinium corymbosum or V. myrtillus (blueberry leaves), Rubis idaeus or R. strigosus (Raspberry leaves), Mentha x piperita (peppermint), Matricaria recutita and Matricaria chamomilla or Chamomilla recutitia (chamomile), Pimpinella anisum (anise), Carum carvi (caraway), Coriandrum sativum (coriander), Foeniculum vulgare (fennel), Acorus calamus (calamus), Curcuma domestica or C. longa, C. zanthorrhiza, C. zedoaria (turmeric), Peumus boldus (boldo), Taraxacum officinale (dandelion), and Glycyrrhiza glabra or G. glabra (licorice)

A variety of minerals may also be added. Zinc oxide, zinc gluconate, zinc sulfate, zinc citrate and/or zinc as aminoate complex may be used in lieu of or in addition to other minerals. Zinc enhances the body's immune system and this enhancement is believed to be beneficial against various pathogens including, it is further believed, Helicobacter pylon (previously called Campylobacter pyloi), the bacteria commonly associated with gastritis and the formation of ulcers. In addition to assisting in the healing of the body, these minerals also provide supplementary amounts of calcium, magnesium and zinc, all necessary metals to maintain body health and metabolism. Furthermore, the minerals and vitamins promote easier and more complete absorption of nutrients by the body, while not changing the pH of the digestive tract which is a common reaction to inorganic salts.

Preferably, the fiber is one or more sources of edible fiber. As that term is used herein, “edible fiber” refers to any source of roughage that is capable of being ingested and processed without harm by animals, in particular humans. Fiber from whatever source is therefore suitable, especially plant matter, such as bran from oats, wheat, corn and rice etc., as well as cellulosic, pectin and gum materials. Husk material may also be preferred. The term “fiber” includes both “soluble” and “insoluble” fiber.

Sweeteners can be chosen from the list of saccharide material available as the carrier component, or can be different materials from those comprising the carrier material, heretofore described. The sweetener is added primarily to provide the sleep inducing antacid composition with a palatable sweet taste and flavor. Sweeteners can include mono-, di-, tri- and polysaccharide materials, either alone or in combination, and their related oligomers. Invert sugar, sucrose, fructose, maltose, dextrose, polydextrose, polydextrin, glucose (corn syrup), maltodextrin (corn syrup solids) etc. are just some examples of suitable sweeteners. Additional sweeteners include saccharin, aspartame, acesulfame, sucralose, sorbitol, mannitol, maltitol, and xylitol.

The following nonlimiting examples illustrate further aspects of the invention.

### EXAMPLE 1

Preparation of Sleep Inducing Antacid Composition.

A sleep inducing antacid composition was prepared by combining valerian and calcium carbonate. The valerian was commercially available as valerian root capsule from Sundown Herbals. Each capsule contained 100 mg valerian extract root that is standardized to 0.8% valerenic acid (0.8 mg); and valerian Indian root (430 mg). Two capsules of valerian were administered in a single dosage.

The calcium carbonate was commercially available as MAALOX® antacid, calcium carbonate chewable tablets from Novartis. Each tablet contained 600 mg. of calcium carbonate. The tablet also contained aspartame, colloidal silicon dioxide, crosarmelose sodium, dextrose, magnesium stearate, maltodextrin, mannitol, and pregelatinized starch. One tablet of calcium carbonate was administered.

### EXAMPLE 2

Evaluation of Sleep Inducing Antacid Composition Prepared in Example 1.

The sleep inducing antacid composition prepared in Example 1 was administered to three people experiencing excess gastrointestinal acidity. Prior to ingesting the sleep inducing antacid composition and approximately 15 to 20 minutes after ingesting the sleep inducing antacid composition, each person recorded (1) the amount of excess gastrointestinal acidity on a scale of 1 to 5 wherein 5 represented a high level of excess gastrointestinal acidity and 1 represented no excess gastrointestinal acidity; and (2) their level of drowsiness on a scale of 1 to 5 wherein 5 represented extreme alertness and 1 represented lethargy. The test results are summarized in Table I.

TABLE I

| Excess | Excess | | |
|---|---|---|---|
| Gastrointestinal | Gastrointestinal | | |
| Acidity | Acidity | Drowsiness Level | Drowsiness Level |
| (Before) | (After) | (Before) | (After) |
| 5 | 1 | 5 | 1 |
| 4 | 1 | 4 | 1 |
| 5 | 2 | 4 | 1 |

The test results in Table I clearly show that the sleep inducing antacid composition of the invention reduced excess gastrointestinal acidity in each of the three people within about 15 minutes and increased the level of drowsiness in each of the three people within about 15 minutes after ingesting the sleep inducing antacid composition.

### EXAMPLE 3

Preparation of Sleep Inducing Antacid Composition.

An sleep inducing antacid composition was prepared according to the formula set forth in Example 1, except that 1 capsule of valerian was administered instead of two capsules.

### EXAMPLE 4

Evaluation of Sleep Inducing Antacid Composition Prepared in Example 3.

The sleep inducing antacid composition prepared in Example 1 was administered to three people experiencing excess gastrointestinal acidity. Prior to ingesting the sleep inducing antacid composition and approximately 15 to 20 minutes after ingesting the sleep inducing antacid composition, each person recorded (1) the amount of excess gastrointestinal acidity on a scale of 1 to 5 wherein 5 represented a high level of excess gastrointestinal acidity and 1 represented no excess gastrointestinal acidity; and (2) their level of drowsiness on a scale of 1 to 5 wherein 5 represented extreme alertness and 1 represented lethargy. The test results are summarized in Table II.

TABLE II

| Excess | Excess | | |
|---|---|---|---|
| Gastrointestinal | Gastrointestinal | | |
| Acidity | Acidity | Drowsiness Level | Drowsiness Level |
| (Before) | (After) | (Before) | (After) |
| 5 | 1 | 5 | 2 |
| 4 | 1 | 5 | 1 |
| 5 | 2 | 4 | 2 |

The test results in Table II clearly show that the sleep inducing antacid composition of the invention reduced excess gastrointestinal acidity in each of the three people within about 15 minutes and increased the level of drowsiness in each of the three people within about 15 minutes after ingesting the sleep inducing antacid composition.

The advantage of the sleep inducing antacid compositions are that they promote drowsiness and provide relief from excess gastrointestinal acidity.

While the invention has been described with particular reference to certain embodiments thereof, it will be understood that changes and modifications may be made by those of ordinary skill within the scope and spirit of the following claims:

What is claimed is:

1. An orally administered sleep inducing antacid composition comprising from about 100 mg to about 2000 mg of at least one antacid, and about 300 mg to about 1000 mg of at least one sleep inducing compound wherein the amount of sleep inducing compound is based on a concentrated extract containing not less than 0.5% of the essential oil of the respective sleep inducing compound.

2. An orally administered sleep inducing antacid composition comprising from about 200 mg to about 1200 mg of at least one antacid, and about 400 mg to about 860 mg of at least one sleep inducing compound wherein the amount of sleep inducing compound is based on a concentrated extract containing not less than 0.5% of the essential oil of the respective sleep inducing compound.

3. An orally administered sleep inducing antacid composition comprising from about 400 mg to about 800 mg of at least one antacid, and about 430 mg to about 600 mg of at least one sleep inducing compound wherein the amount of sleep inducing compound is based on a concentrated extract containing not less than 0.5% of the essential oil of the respective sleep inducing compound.

4. The composition according to claim 1 wherein the antacid is selected from the group consisting of aluminum carbonate, aluminum hydroxide (or as aluminum hydroxide-hexitol stabilized polymer, aluminum hydroxide-magnesium hydroxide codried gel, aluminum hydroxide-magnesium trisilicate codried gel, aluminum hydroxide-sucrose powder hydrated), aluminum phosphate, aluminum hydroxy carbonate, dihydroxy aluminum sodium carbonate, aluminum magnesium glycinate, dihydroxy aluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, magnesium trisilicate, sodium bicarbonate, potassium bicarbonate, glycine, dried milk solids, sodium carbonate, potassium carbonate, sodium potassium tartrate, and combinations thereof.

5. The composition according to claim 4 wherein the antacid is selected from the group consisting of aluminum hydroxide, calcium carbonate, magnesium carbonate, and magnesium hydroxide.

6. The composition according to claim 1 wherein the sleep inducing compound is selected from the group consisting of herbal compounds, nonherbal compounds, and combinations thereof.

7. The composition according to claim 6 wherein the sleep inducing compound is selected from the group consisting of melatonin, L-tryptophan, Amanita Muscaria (aga), Apium Graveolens (celery), Aquilegia Vulgaris (columbine), Artemisia Vulgaris (mugwort), Atropa Belladonna (belladonna), Avena Sativa (oats), Ballota Nigra (horehound), Betonica Officinalis (wood betony), Culluna Vulgaris (heather), Cannabis Sativa (marijuana), Capsella Bursa Pastoris (shepherd's purse), Chamaemelum Nobile (Roman chamomile), Cinnamomum Camphora (camphor tree), Citrus Aurantium (bitter orange), Convallaria Majalis (lily-of-the-valley), Corydalis Caba (corydalis), Cyclamen Europaeum (cyclamen), Cypripedium Calceolus (nerve root), Cytisus Scoparius (broom), Drimia Maritima (squill), Eschscholtzia Californica (California poppy), Fragaria Vesca (strawberry leaf), Galium Odorata (woodruff), Humulus Lupulus (hops), Hypericum Perforatum (St. John's wort), Ilex Paraguariensis (mate), Lavandula Angustifolia (lavender), Leonurus Cardiaca (motherwort), Lycopus Virginicus (bugleweed), Matricaria chamomile L. (German chamomile), Matricaria recutita L. (Hungarian chamomile), Melissa Officinalis (lemon balm), Nepeta Cataria (catnip), Paris Quadrifolia (herb Paris), Passiflora Incarnata (passion flower), Piper Methysticum (kava-kava), Piscidia Piscipula (Jamaica dogwood), Primula Elatior (primrose), Prunus Serotina (black cherry), Rauwolfia Serpentina (rauwolfia), Scutellaria Lateriflora (scullcap), Selenicereus Grandiflorus (night-blooming cereus), Strychnos Nux Vomica (nux vomica), Syzygium Cumini (jambolan), Valeriana Officinalis (valerian), Verbena Officinalis (vervain), Veronica Officinalis (speedwell), Viscum Album (mistletoe), and combinations thereof.

8. The composition according to claim 7 wherein the sleep inducing compound is selected from the group consisting of Apium Graveolens (celery), Avena Sativa (oats) Chamaemelum Nobile (Roman chamomile), Convallaria Majalis (lily-of-the-valley), Cypripedium Calceolus (nerve root), Hypericum Perforatum (St. John's wort) Lavandula Angustifolia (lavender), Mattricaria Chamomile L. (German chamomile), Nepeta Cataria (catnip), Piper Methysticum (kava-kava), and Valeriana Officinalis (valerian).

9. The composition according to claim 8 wherein the sleep inducing compound is valerian.

10. The composition according to claim 9 wherein the sleep inducing compound is selected from the group consisting of valerenic acid, hydroxyvalerenic acid, acetoxyvalerenic acid, valerenal, valeranone, kessyl glycol, didrovaltrate, valtrate, and isovaltrate.

11. The composition according to claim 8 wherein the sleep inducing compound is kava-kava.

12. The composition according to claim 11 wherein the sleep inducing compound is selected from the group consisting of methysticin, dihydromethysticin, kawain, dihydrokawain, yangonin, marindinin, and desmethoxyyangonin.

13. The composition according to claim 1 further comprising an ingredient selected from the group consisting of a vitamin, botanical, supplement, mineral, trace element, amino acid, antiflatulent, herb, fiber, flavorant, enzyme, filler, buffer, colorant, dye, sweetener, pharmaceutical active compound, antioxidant, food processing aid, carrier material, medicament, preservative, electrolyte, glidant, disintegrate, lubricant, and combinations thereof.

14. The composition according to claim 13 wherein the botanical is selected from the group consisting of Zingiber Officinale Roscoe (ginger), Tiana Lutea (gentianaceae or gentian), Centaurium Erythrae Rafn (centaury), Swertia Chirata (bitterstick), Menyanthes Trifoliata (bogbean), Artermisia Absinthium (worm wood), Rubis Fruiticousus (blackberry leaves or fruit), Rubis family (blackberry root), Vaccinium Corymbosum or V. Myrtillus (blueberry leaves), Rubis Idaeus or R. Strigosus (raspberry leaves), Mentha x Piperita (peppermint), Matricaria Recutita and Matricaria Chamomilla or Chamomilia Recutitia (chamomile), Pimpinella Anisum (anise), Carum Carvi (caraway), Coriandrum Sativum (coriander), Foeniculum Vulgare (fennel), Acorus Calamus (calamus), Curcuma Domestica or C. Longa, C. Zanthorrhiza, C. Zedoaria (turmeric), Peumus Boldus (boldo), Taraxacum Officinale (dandelion), Glycyrrhiza Glabra or G. Glabra (licorice), and combinations thereof.

15. A method to induce drowsiness in animals including humans comprising orally administering to the animal a sleep inducing antacid composition comprising from about 100 mg to about 2000 mg of at least one antacid, and about 300 mg to about 1000 mg of at least one sleep inducing compound wherein the amount of sleep inducing compound is based on a concentrated extract containing not less than 0.5% of the essential oil of the respective sleep inducing compound.
